(19) United States
(12) Patent Application Publication
(10) Pub. No.: US 2007/0185135 A1
(43) Pub. Date: Aug. 9, 2007

(54) Drug-Polymer Conjugates

(75) Inventors: Laurence I. Wu, Tainan (TW); Bryan T. H. Wu, Taipei City (TW); Kuo-Hsi Kao, Taipei County (TW); Li-Ming Shen, Taipei (TW); Kelly Teng, San Diego, CA (US)

Correspondence Address: OCCHIUTI ROHLICEK & TSAO, LLP, 10 FAWCETT STREET, CAMBRIDGE, MA, 02138, US

(73) Assignee: PHARMAESSENTIA CORP.

(21) Appl. No.: 11/617,868

(22) Filed: Dec. 29, 2006

Related U.S. Application Data
(60) Provisional application No. 60/755,459, filed on Dec. 30, 2005.

(51) Int. Cl. C07D 239/42

(52) U.S. Cl. 514/256

(57) ABSTRACT

This invention relates to a polypeptide-polymer conjugate that includes a polypeptide moiety, a polyalkylene oxide moiety, a linker connecting the polypeptide moiety with the polyalkylene oxide moiety, a first linkage between the polypeptide moiety and the linker, and a second linkage between the polyalkylene oxide moiety and the linker.

### CROSS-REFERENCE TO RELATED APPLICATION

This application claims priority under 35 U.S.C. § 119(e) to U.S. Provisional Patent Application Ser. No. 60/755,459, filed Dec. 30, 2005, and the contents of which are hereby incorporated by reference.

### BACKGROUND

Two major drug delivery approaches have been investigated to improve pharmacodynamic and pharmacokinetic properties of therapeutic drug molecules. One is to modify the drug molecule itself (e.g., by pegylation) and the other is to change the drug formulation (e.g., by using liposomal preparations). In either case, it is desirable to develop a drug delivery mechanism that provides a prolonged pharmacologic activity, decreased adverse effects, increased patient compliance, and improved life quality of patients.

### SUMMARY

This invention is based on the concept that a therapeutic polypeptide molecule can be coupled to a polymer molecule to form a single drug entity, i.e., a polypeptide-polymer conjugate, with improved efficacy.

In one aspect, this invention features a polypeptide-polymer conjugate that includes a polypeptide moiety, a polyalkylene oxide moiety, a linker connecting the polypeptide moiety with the polyalkylene oxide moiety, a first linkage between the polypeptide moiety and the linker; and a second linkage between the polyalkylene oxide moiety and the linker. The polypeptide moiety can contain a human interferon-α moiety (i.e., a native or modified moiety retaining interferon-α activities) and 1-6 (e.g., 1-4) additional amino acid residues at the N-terminus of the human interferon-α moiety. Examples include -Ser-Gly-IFN, -Gly-Ser-IFN, -Met-Met-IFN, -Met-His-IFN, -Pro-IFN, and -Gly-Met-IFN, in which IFN is a human interferon-α_{2b} moiety. The interferon-α moiety can include a cysteine residue at the N-terminus. The polypeptide moiety can also include an interferon-β moiety or a granulocyte colony-stimulating factor. The polyalkylene oxide moiety can contain 1-20,000 C₁-C₈ alkylene oxide repeating units. Examples of a polyalkylene oxide moiety include polyethylene oxide moieties containing 5-10,000 repeating units, such as a polyethylene oxide moiety having a number average molecular weight of 20,000 Daltons. The linker can be C₁-C₈ alkylene, C₁-C₈ heteroalkylene, C₃-C₈ cycloalkylene, C₃-C₈ heterocycloalkylene, arylene, heteroarylene, aralkylene, or —Ar—X—(CH₂)ₙ—, in which Ar can be arylene (e.g., phenylene) or heteroarylene, X can be O, S, or N(R), R being H or C₁-C₁₀ alkyl, and n can be 1-10. Each of the first and second linkages, independently, can be a carboxylic ester, carbonyl, carbonate, amide, carbamate, urea, ether, thio, sulfonyl, sulfinyl, amino, imino, hydroxyamino, phosphonate, or phosphate group. An example of the just-described drug-polymer conjugate is 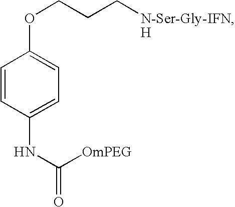
in which mPEG is a methoxy-capped polyethylene oxide moiety.

A polyalkylene oxide moiety refers to a linear, branched, or star-shaped moiety. It is either saturated or unsaturated and either substituted or unsubstituted. Examples of polyalkylene oxide moieties include polyethylene oxide, polypropylene oxide, polyisopropylene oxide, polybutenylene oxide, and copolymers thereof. Other polymers such as dextran, polyvinyl alcohols, polyacrylamides, or carbohydrate-based polymers can also be used to replace polyalkylene oxide moiety, as long as they are not antigenic, toxic, or eliciting immune response.

A linker extends from a polyalkylene oxide moiety and facilitates coupling the polypeptide moiety to the polyalkylene oxide moiety.

A polypeptide moiety can include a modified polypeptide drug as long as at least some of its pharmaceutical activity is retained. Examples of such a therapeutic polypeptide moiety include modified polypeptide molecules containing one or more additional amino acid residues at the N-terminus or modified polypeptide molecules containing one or more substitutions for the amino acid residues within their primary protein sequences.

The polypeptide moiety can be released in vivo (e.g., through hydrolysis) under enzymatic actions by cleaving the linkage between the polypeptide moiety and the linker or the linkage between the polyalkylene oxide moiety and the linker. Examples of enzymes involved in cleaving linkages in vivo include oxidative enzymes (e.g., peroxidases, amine oxidases, or dehydrogenases), reductive enzymes (e.g., keto reductases), and hydrolytic enzymes (e.g., proteases, esterases, sulfatases, or phosphatases). A polypeptide-polymer conjugate of the invention can also be effective without cleaving the therapeutic polypeptide moiety from the polypeptide-polymer conjugate in vivo.

The term “alkyl” refers to a monovalent, saturated, linear or branched, non-aromatic hydrocarbon moiety, such as —CH₃ or —CH(CH₃)₂. The term “alkenyl” refers to a linear or branched hydrocarbon moiety that contains at least one double bond, such as —CH═CH—CH₃. The term “alkynyl” refers to a linear or branched hydrocarbon moiety that contains at least one triple bond, such as —C≡C—CH₃. The term “cycloalkyl” refers to a saturated, cyclic hydrocarbon moiety, such as a cyclopropyl. The term “cycloalkenyl” refers to a non-aromatic, cyclic hydrocarbon moiety that contains at least one ring double bond, such as cyclohexenyl. The term “heterocycloalkyl” refers to a saturated, cyclic moiety having at least one ring heteroatom (e.g., N, O, or S), such as 4-tetrahydropyranyl. The term “heterocycloalkenyl” refers to a non-aromatic, cyclic moiety having at least one ring heteroatom (e.g., N, O, or S) and at least one ring double bond, such as pyranyl. The term “aryl” refers to a hydrocarbon moiety having one or more aromatic rings. Examples of aryl moieties include phenyl (Ph), naphthyl, pyrenyl, anthryl, and phenanthryl. The term “heteroaryl” refers to a moiety having one or more aromatic rings that contain at least one ring heteroatom (e.g., N, O, or S). Examples of heteroaryl moieties include furyl, fluorenyl, pyrrolyl, thienyl, oxazolyl, imidazolyl, thiazolyl, pyridyl, pyrimidinyl, quinazolinyl, quinolyl, isoquinolyl, and indolyl. The term “alkylene” refers to a divalent, saturated, linear or branched, non-aromatic hydrocarbon moiety, such as —CH₂—. The term “heteroalkylene” refers to an alkylene moiety having at least one heteroatom (e.g., N, O, or S), such as —CH₂OCH₂—. The term “cycloalkylene” refers to a divalent, saturated cyclic hydrocarbon moiety, such as cyclohexylene. The term “heterocycloalkylene” refers to a divalent, saturated, non-aromatic cyclic moiety having at least one ring heteroatom, such as 4tetrahydropyranylene. The term “arylene” refers to a divalent hydrocarbon moiety having one or more aromatic rings. Examples of an aryl moiety include phenylene and naphthylene. The term “heteroarylene” refers to a divalent moiety having one or more aromatic rings that contain at least one ring heteroatom. Examples of a heteroarylene moiety include furylene and pyrrolylene. The term “aralkylene” refers to a divalent alkyl moiety substituted with aryl or heteroaryl, in which one electron is located on the alkyl moiety and the other electron is located on aryl or heteroaryl. Examples of a aralkylene moiety include benzylene or pyridinylmethylene.

Alkyl, alkenyl, alkynyl, cycloalkyl, cycloalkenyl, heterocycloalkyl, heterocycloalkenyl, aryl, heteroaryl, alkylene, heteroalkylene, cycloalkylene, heterocycloalkylene, arylene, heteroarylene, and aralkylene mentioned herein include both substituted and unsubstituted moieties. Examples of substituents for cycloalkylene, heterocycloalkylene, arylene, heteroarylene, and aralkylene include C₁-C₁₀ alkyl, C₂-C₁₀ alkenyl, C₂-C₁₀ alkynyl, C₃-C₈ cycloalkyl, C₅-C₈ cycloalkenyl, C₁-C₁₀ alkoxy, aryl, aryloxy, heteroaryl, heteroaryloxy, amino, C₁-C₁₀ alkylamino, C₁-C₂₀ dialkylamino, arylamino, diarylamino, hydroxyamino, alkoxyamino, C₁-C₁₀ alkylsulfonamide, arylsulfonamide, hydroxy, halogen, thio, C₁-C₁₀ alkylthio, arylthio, cyano, nitro, acyl, acyloxy, carboxyl, and carboxylic ester. Examples of substituents for alkyl, alkylene, and heteroalkylene include all of the above substitutents except C₁-C₁₀ alkyl. Cycloalkylene, heterocycloalkylene, arylene, and heteroarylene can also be fused with cycloalkyl, heterocycloalkyl, aryl or heteroaryl.

In another aspect, this invention features a polypeptide-polymer conjugate that includes a polypeptide moiety, a polyalkylene oxide moiety, a linker connecting the polypeptide moiety with the polyalkylene oxide moiety, a first linkage between the polypeptide moiety and the linker, and a second linkage between the polyalkylene oxide moiety and the linker. The polyalkylene oxide moiety can contain 1-20,000 C₁-C₈ alkylene oxide repeating units. The linker can be —Ar—X—(CH₂)ₙ—, in which Ar can be arylene or heteroarylene, X can be O, S, or N(R), R being H or C₁-C₁₀ alkyl, and n can be 10. Each of the first and second linkages, independently, can be a carboxylic ester, carbonyl, carbonate, amide, carbamate, urea, ether, thio, sulfonyl, sulfinyl, amino, imino, hydroxyamino, phosphonate, or phosphate group.

In another aspect, this invention features a compound of formula (I): 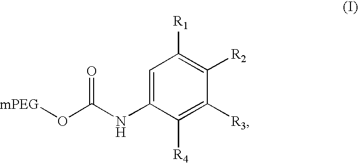
In formula (I), mPEG is a methoxy-capped polyethylene oxide moiety; one of R₁, R₂, R₃, and R₄ is C₁-C₁₀ alkyl substituted with CHO; and each of the other R₁, R₂, R₃, and R₄, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl. A subset of the compounds of formula (I) are those in which R₂ or R₃ is propyl substituted with CHO or butyl substituted with CHO.

In another aspect, this invention features a polypeptide that includes an interferon-α moiety (e.g., a human interferon-α_{2b} moiety) and 1-6 additional amino acid residues at the N-terminus of the interferon-α moiety. Examples include Ser-Gly-IFN, Gly-Ser-IFN, Met-Met-IFN, Met-His-IFN, Pro-IFN, and Gly-Met-IFN, in which IFN is a human interferon-α_{2b} moiety. The interferon-α moiety can also be a wild type interferon-α moiety (e.g., a wild type human interferon-α_{2b} moiety).

In another aspect, this invention features a method for treating various diseases, such as hepatitis B virus infection, hepatitis C virus infection, and cancer (e.g., hairy-cell leukemia or Kaposi sarcoma). The method includes administering to a subject in need thereof an effective amount of one or more polypeptide-polymer conjugates described above. The term “treating” or “treatment” refers to administering one or more polypeptide-polymer conjugates to a subject, who has an above-mentioned disease, a symptom of it, or a predisposition toward it, with the purpose to confer a therapeutic effect, e.g., to cure, relieve, alter, affect, ameliorate, or prevent the above-mentioned disease, the symptom of it, or the predisposition toward it.

This invention also encompasses a pharmaceutical composition that contains an effective amount of at least one of the above-mentioned polypeptide-polymer conjugates and a pharmaceutically acceptable carrier.

The polypeptide-polymer conjugates described above include the compounds themselves, as well as their salts, prodrugs, and solvates, if applicable. A salt, for example, can be formed between an anion and a positively charged group (e.g., amino) on a polypeptide-polymer conjugate. Suitable anions include chloride, bromide, iodide, sulfate, nitrate, phosphate, citrate, methanesulfonate, trifluoroacetate, and acetate. Likewise, a salt can also be formed between a cation and a negatively charged group (e.g., carboxylate) on a polypeptide-polymer conjugate. Suitable cations include sodium ion, potassium ion, magnesium ion, calcium ion, and an ammonium cation such as tetramethylammonium ion. Examples of prodrugs include esters and other pharmaceutically acceptable derivatives, which, upon administration to a subject, are capable of providing active polypeptide-polymer conjugates. A solvate refers to a complex formed between an active polypeptide-polymer conjugate and a pharmaceutically acceptable solvent. Examples of pharmaceutically acceptable solvents include water, ethanol, isopropanol, ethyl acetate, acetic acid, and ethanolamine.

Also within the scope of this invention is a composition containing one or more of the polypeptide-polymer conjugates described above for use in treating various diseases mentioned above, and the use of such a composition for the manufacture of a medicament for the just-mentioned treatment.

The details of one or more embodiments of the invention are set forth in the description below. Other features, objects, and advantages of the invention will be apparent from the description and from the claims.

### DETAILED DESCRIPTION

This invention relates to polypeptide-polymer conjugates in which a therapeutic polypeptide moiety is coupled to at least one polymer molecule.

Polypeptide-polymer conjugates can be prepared by synthetic methods well known in the chemical art. For example, a linker molecule containing a functional group (e.g., an phenylamino group) can be first coupled to a methoxy-capped polyethylene glycol (mPEG) polymer containing a hydroxy end group through a carbamate linkage to form a linker-polymer conjugate. Subsequently, a therapeutic polypeptide molecule (e.g., human interferon-α_{2b}) containing another functional group (e.g., an amino group) can be coupled to the above linker-polymer conjugate after converting the other end group on the linker-polymer conjugate into an aldehyde group. To couple with a linker molecule, the mPEG polymer can be functionalized with groups such as succinimidyl ester, p-nitrophenol, succinimidyl carbonate, tresylate, maleimide, vinyl sulfone, iodoacetamide, biotin, phospholipids, or fluroescein. As another example, a therapeutic polypeptide molecule (e.g., human interferon-α_{2b}) can be first modified by introducing 1-6 additional amino acid residues at its N-terminus through recombinant technology. The modified human interferon-α_{2b} molecule can then be coupled to a methoxy-capped polyethylene glycol moiety containing a linker at one end. The coupling reaction can be achieved by modifying the linker to form a suitable function group (e.g., an aldehyde group) and then reacting that functional group on the linker with a functional group on the modified human interferon-α_{2b} molecule (e.g., a terminal amino group).

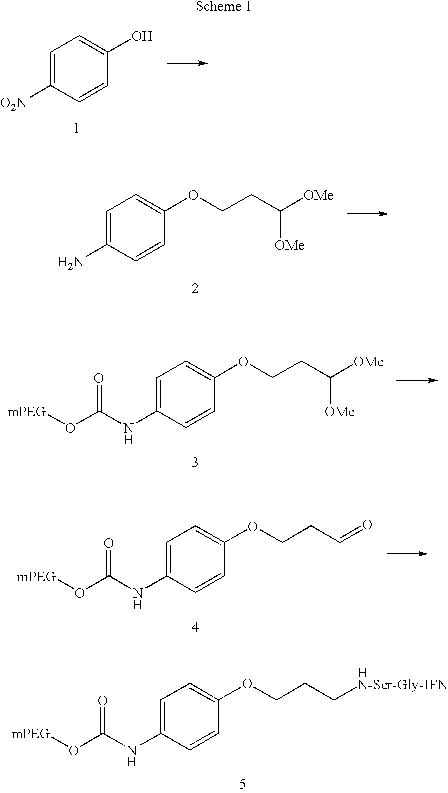

Scheme 1 above illustrates an example of the preparation of one of the polypeptide-polymer conjugate described above. 4-Nitrophenol 1 is first converted into linker molecule 2 in four chemical transformations: (a) alkylation of the hydroxyl group with 3-chloropropan-1-ol; (b) oxidation of the terminal hydroxyl group to an aldehyde group; (c) protecting the aldehyde group by forming a dimethyl acetal group; (d) reduction of the nitro group to an amino group. Methoxy-capped polyethylene glycol (mPEG) polymer is then coupled to linker molecule 2 by using N,N-disuccinimidyl carbonate to produce linker-polymer conjugate 3. The dimethyl acetal protecting group in linker-polymer conjugate 3 is subsequently removed to give linker-polymer conjugate 4 containing an aldehyde group, which is then coupled with a modified human interferon-α_{2b} molecule, Ser-Gly-IFN, to form the polypeptide-polymer conjugate 5.

The chemicals used in the above-described synthetic route may include, for example, solvents, reagents, catalysts, protecting group and deprotecting group reagents. The methods described above may additionally include steps, either before or after the steps described specifically herein, to add or remove suitable protecting groups in order to ultimately allow for synthesis of a polypeptide-polymer conjugate. In addition, various synthetic steps may be performed in an alternate sequence or order to give the desired polypeptide-polymer conjugates. Synthetic chemistry transformations and protecting group methodologies (protection and deprotection) useful in synthesizing applicable polypeptide-polymer conjugates are known in the art and include, for example, those described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

A polypeptide-polymer conjugate thus synthesized can be further purified by a method such as column chromatography or high-pressure liquid chromatography.

The polypeptide-polymer conjugates mentioned herein may contain a non-aromatic double bond and one or more asymmetric centers. Thus, they can occur as racemates and racemic mixtures, single enantiomers, individual diastereomers, diastereomeric mixtures, and cis- or trans- isomeric forms. All such isomeric forms are contemplated.

One aspect of this invention relates to a method of administering an effective amount of one or more of the above-described polypeptide-polymer conjugates for treating various diseases. Specifically, a disease can be treated by administering one or more of the above-described polypeptide-polymer conjugates in an amount that is required to confer a therapeutic effect to a subject, who has a disease, a symptom of such a disease, or a predisposition toward such a disease, with the purpose to confer a therapeutic effect, e.g., to cure, relieve, alter, affect, ameliorate, or prevent the disease, the symptom of it, or the predisposition toward it. Such a subject can be identified by a health care professional based on results from any suitable diagnostic method.

Also within the scope of this invention is a pharmaceutical composition contains an effective amount of at least one of the polypeptide-polymer conjugates described above and a pharmaceutical acceptable carrier. Effective doses will vary, as recognized by those skilled in the art, depending on, e.g., the rate of hydrolysis of a polypeptide-polymer conjugate, the therapeutic polypeptide moiety in a polypeptide-polymer conjugate, the molecular weight of the polymer, the types of diseases treated, route of administration, excipient usage, and the possibility of co-usage with other therapeutic treatment.

To practice the method of the present invention, a composition having one or more of the above-mentioned polypeptide-polymer conjugates can be administered parenterally, orally, nasally, rectally, topically, or buccally. The term “parenteral” as used herein refers to subcutaneous, intracutaneous, intravenous, intramuscular, intraarticular, intraarterial, intrasynovial, intrasternal, intrathecal, intralesional, intraperitoneal, intratracheal or intracranial injection, as well as any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or diglycerides). Fatty acid, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, or carboxymethyl cellulose or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions, and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art. A composition having one or more of the above-described polypeptide-polymer conjugates can also be administered in the form of suppositories for rectal administration.

A pharmaceutically acceptable carrier is routinely used with one or more active above-mentioned polypeptide-polymer conjugates. The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of an above-mentioned compound. Examples of other carriers include colloidal silicon oxide, magnesium stearate, cellulose, sodium lauryl sulfate, and D&C Yellow # 10.

The example below is to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### EXAMPLE 1

### Preparation of mPEG Aldehydes A-D

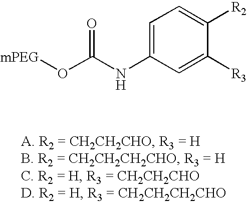 Preparation of mPEG Aldehyde A:
Step A: Preparation of 3-(4-nitrophenoxy)propan-1-ol

3-Chloropropan-1-ol (160 g, 1.69 mol) was added to a solution containing 4-nitrophenol (329 g, 2.37 mol) and KOH (151 g, 2.70 mol) in 1.4 L of a 1:1 ethanol-water mixture. This mixture was heated at reflux for 60 hours, cooled to room temperature, poured into a 1 N aqueous NaOH solution (2.0 L), and extracted with dichloromethane (2×1.2 L). The organic extracts were combined, washed with a 1 N aqueous NaOH solution (1.0 L) and with brine, dried over anhydrous MgSO₄, and concentrated in vacuo to give 3-(4-nitrophenoxy)propan-1-ol (273 g, 82%) as a yellowish solid. ¹H NMR (400 MHz, CDCl₃) δ 8.16 (d, J=9.2 Hz, 2H), 6.94 (d, J=9.2 Hz, 2H), 4.20 (t, J=6.0 Hz, 2H), 3.87-3.83 (m, 2H), 2.10-2.04 (m, 2H), 1.87 (t, J=4.0 Hz, 1H); ¹³C NMR (100 MHz, CDCl₃) δ 163.9, 141.2, 125.8, 114.3, 65.8, 59.1, 31.7; GC-MS (m/z) calcd for C₉H₁₁NO₄: 197.2, found: 197, 139, 123, 109.

Step B. Preparation of 3-(4-nitrophenoxy)propanal

A mixture of NaBr (18.6 g, 181.2 mmol) and TEMPO (0.85 g, 5.4 mmol) in dichloromethane (290 mL) was added to 3-(4-nitrophenoxy)propan-1-ol (35.7 g, 181.2 mmol) in a cold solution of NaOCl (240 mL, as 1:1 mixture of water and a 13 wt % aqueous NaOCl solution) at 0° C. over a period of 30 minutes. When the addition was complete, the mixture became pale yellow and was stirred at 0° C. for 1 hour. After the resulting mixture was partitioned, the organic layer was washed with water (300 mL), dried over anhydrous MgSO₄, and concentrated in vacuo to give 3-(4-nitrophenoxy) propanal (31 g, 87%) as a pale yellow liquid. ¹H NMR (400 MHz, CDCl₃) δ 9.93 (s, 1H), 8.24 (d, J=9.2 Hz, 2H), 7.01 (d, J=9.2 Hz, 2H), 4.45 (t, J=6.0 Hz, 2H), 3.05 (t, J=6.0 Hz, 2H); GC-MS (m/z) calcd for C₉H₉NO₄:195.2, found: 195, 167, 139, 109, 93, 65.

Step C: Preparation of 3-(4-nitrophenoxy)propanal dimethyl acetal

AMBERLITE lra-400 (CI) ion exchange resin (30 g) was added to a solution of 3-(4-nitrophenoxy) propanal (30 g, 0.15 mol) in methanol (300 mL). The resulting mixture was stirred at room temperature for 16 hours and filtered through Celite. The filtrate was concentrated in vacuo to give 3-(4-nitrophenoxy)propanal dimethyl acetal (30 g, 80%) as a pale yellow solid. ¹H NMR (400 MHz, CDCl₃) δ 8.17 (d, J=9.2 Hz, 2H), 6.94 (d, J=9.2 Hz, 2H), 4.61 (t, J=6.0 Hz, 1H), 4.13 (t, J=6.4 Hz, 2H), 3.62 (s, 6H), 2.09-2.14 (m, 2H); ¹³C NMR (100 MHz, CDCl₃) δ 163.8, 141.4, 125.8, 114.3, 101.6, 64.8, 53.3, 32.4; GC-MS (m/z) calcd for C₁₁H₁₅NO₃: 241.2, found: 241, 178, 152, 75.

Step D. Preparation of 3-(4-aminophenoxy)propanal dimethyl acetal

Sodium borohydride (15.0 g, 0.39 mol) was added to a cold solution of 3-(4-nitrophenoxy) propanal dimethyl acetal (30.0 g, 0.12 mol) and copper (I) chloride (1.2 g, 12.4 mmol) in ethanol (500 mL). The mixture was heated at 60° C. with stirring for 30 minutes, cooled to room temperature, diluted with water (250 mL), concentrated in vacuo to remove ethanol, and extracted with methyl t-butyl ether or MTBE (3×150 mL). The organic extracts were combined, washed with brine, dried over anhydrous MgSO₄, and concentrated in vacuo to give a crude residue. The crude residue was purified by column chromatography on neutral aluminum oxide using 40% ethyl acetate-hexanes as an eluant to give 3-(4-aminophenoxy) propanal dimethyl acetal (19.5 g, 75%) as a deep purple liquid. ¹H NMR (400 MHz, CDCl₃) δ 6.74 (d, J=8.8 Hz, 2H), 6.66 (d, J=8.8 Hz, 2H), 4.62 (t, J=5.6 Hz, 1H), 3.95 (t, J=6.0 Hz, 2H), 3.35 (s, 6H), 2.01-2.06 (m, 2H); ¹³C NMR (100 MHz, CDCl₃) δ 152.3, 139.1, 116.7, 115.6, 102.1, 64.5, 53.2, 32.8; GC-MS (m/z) calcd for C₁₁H₁₇NO₃: 211.3, found: 211, 148, 109, 75.

Step E: Preparation of mPEG aldehyde A dimethyl acetal

Linear 20 kDa mPEG-OH (60.0 g, 3 mmol) was dissolved in 300 mL of dry dioxane with gentle heating. After the solution was cooled to room temperature, N,N-disuccinimidyl carbonate (5.0 g, 19.5 mmol) and 4-(dimethyl amino)pyridine (2.5 g, 20.4 mmol) were sequentially added. The reaction mixture was stirred at room temperature for 24 hours. 3-(4-aminophenoxy)propanal dimethyl acetal (15.0 g, 71.0 mmol) was then added to the reaction mixture. After this mixture was stirred at room temperature for another 18 hours, MTBE (4.5 L) was added dropwise over a period of 4 hours. The resulting white precipitates were collected and dried under vacuum to yield 59.5 g of the crude product, which was redissolved in dichloromethane (250 mL). Another batch of MTBE (6.0 L) was added dropwise over a period of 4 hours. The white precipitates thus obtained were collected and dried under vacuum to give mPEG Aldehyde A dimethyl acetal (58.0 g, 97%) as a white powder. ¹H NMR (400 MHz, DMSO-d₆) δ 9.54 (br, 1H), 7.35 (d, J=8.8 Hz, 2H), 6.85 (d, J=8.8 Hz, 2H), 4.56 (t, J=5.6 Hz, 1H), 4.17 (t, J=4.4 Hz, 2H), 3.93 (t, J=9.6 Hz, 2H), 3.25 (s, 6H), 3.24 (s, 3H), 1.93-1.97 (m, 2H).

Step F: Preparation of mPEG aldehyde A

mPEG aldehyde A dimethyl acetal (55.0 g, 2.75 mmol) was dissolved in a buffer solution (600 mL, citric acid-HCl-NaCl, pH=2). This solution was stirred at room temperature for 20 hours and extracted with dichloromethane (6×200 mL). The organic extracts were combined, washed with brine, dried over anhydrous Na₂SO₄, concentrated in vacuo to approximately 350 mL in volume. MTBE (6.0 L) was then added dropwise over a period of 6 hours. The resulting white precipitates were collected and dried under vacuum to give mPEG Aldehyde A (52.0 g, 95%) as a white powder. ¹H NMR (400 MHz, DMSO-d₆) δ 9.73 (s, 1H), 9.56 (br, 1H), 7.36 (d, J=8.8 Hz, 2H), 6.86 (d, J=8.8 Hz, 2H), 4.23 (t, J=6.0 Hz, 2H), 4.17 (t, J=4.8 Hz, 2H), 3.32 (s, 3H), 2.8-2.87 (m, 2H).

Preparation of mPEG aldehyde B:

Step A: Preparation of 4-(4-nitrophenoxy)butan-1-ol

p-Nitrofluorobenzene (10.0 g, 70.7 mmol) was added slowly to a mixture of 1,4-butanediol (31.9 g, 354 mmol) and potassium hydroxide (5.0 g, 89.1 mmol) at room temperature over a period of 15 minutes. The mixture was stirred at room temperature for 1 hour. It was then poured into water and extracted with dichloromethane. The organic extract was washed with brine, dried over anhydrous MgSO₄, and concentrated in vacuo to give a crude product. The crude product was recrystallized from ethyl acetate-hexanes to give 4-(4-nitrophenoxy)butan-1-ol (9.6 g, 64%) as a white solid. ¹H NMR (400 MHz, CDCl₃) δ 8.22 (d, J=8.8 Hz, 2H), 6.98 (d, J=8.8 Hz, 2H), 4.14 (t, J=6.0 Hz, 2H), 3.80-3.75 (m, 2H), 2.00-1.94 (m, 2H), 1.83-1.76 (m, 2H), 1.65-1.48 (br, 1H); ¹³C NMR (100 MHz, CDCl₃) δ 164.0, 141.4, 125.9, 114.4, 68.6, 62.3, 29.0, 25.5; GC-MS (m/z) calcd for C₁₀H₁₃NO₄: 211.2, found: 211, 139, 123, 109, 73, 55.

Step B: Preparation of 4-(4-nitrophenoxy)butanal

4-(4-Nitrophenoxy)butanal was obtained as a white solid in 81% yield from 4-(4-nitrophenoxy)butan-1-ol using the method described in Step B for preparing mPEG aldehyde A. ¹H NMR (400 MHz, CDCl₃) δ 9.86 (s, 1H), 8.17 (d, J=8.8 Hz, 2H), 6.94 (d, J=8.8 Hz, 2H), 4.12 (t, J=6.0 Hz, 2H), 2.71 (t, J=6.0 Hz, 2H), 2.18 (m, 2H); ¹³C NMR (100 MHz, CDCl₃) δ 200.3, 162.8, 140.5, 124.9, 113.5, 66.7, 39.3, 20.7; GC-MS (m/z) calcd for C₁₀H₁₁NO₄: 209.2, found: 209, 139, 123, 109, 71.

Step C. Preparation of 4-(4-nitrophenoxy)butanal dimethyl acetal

4-(4-Nitrophenoxy)butanal dimethyl acetal was obtained as a pale yellow solid in 82% yield from 4-(4-nitrophenoxy)butanal using the method described in step C for preparing mPEG aldehyde A. ¹H NMR (400 MHz, CDCl₃) δ 8.19 (d, J=8.8 Hz, 2H), 6.96 (d, J=8.8 Hz, 2H), 4.62 (t, J=5.6 Hz, 1H), 4.10 (t, J=5.6 Hz, 2H), 3.37 (s, 6H), 1.90-1.93 (m, 2H), 1.85-1.81 (m, 2H); ¹³C NMR(100 MHz, CDCl₃) δ 163.9, 141.3, 125.8, 114.3, 104.0, 68.3, 52.9, 28.9, 24.1; GC-MS (m/z) calcd for C₁₂H₁₇NO₅: 255.3, found: 255, 224, 192, 117, 75.

Step D: Preparation of 4-(4-aminophenoxy)butanal dimethyl acetal

4-(4-Nitrophenoxy)butanal dimethyl acetal (4.0 g, 15.7 mmol) was dissolved in methanol (40 mL) and hydrogenated in the presence of 10% palladium on carbon (0.4 g) at room temperature for 16 hours. After the mixture was filtered through Celite, the filtrate was concentrated in vacuo to give a crude residue, which was purified by column chromatography on neutral aluminum oxide using 50% ethyl acetate-hexanes as an eluant to give 4-(4-aminophenoxy)butanal dimethyl acetal (2.5 g, 70%) as a deep purple liquid.

¹H NMR (400 MHz, CDCl₃) δ 6.70 (d, J=8.8 Hz, 2H), 6.57 (d, J=8.8 Hz, 2H), 4.40 (t, J=5.6 Hz, 1H), 3.85 (t, J=5.6 Hz, 2H), 3.30 (s, 6H), 1.78-1.73 (m, 4H); ¹³C NMR (100 MHz, CDCl₃) δ 151.6, 139.9, 115.9, 115.3, 104.0, 67.8, 52.4, 28.8, 24.3; GC-MS (m/z) calcd for C₁₂H₁₉NO₃: 225.3, found: 225, 194, 162, 109, 85.

Step E: Preparation of mPEG aldehyde B dimethyl acetal

mPEG aldehyde B dimethyl acetal was obtained as a white powder in 93% yield from linear 20 kDa mPEG-OH and 4-(4-aminophenoxy)butanal dimethyl acetal using the method described in Step E for preparing mPEG aldehyde A. ¹H NMR (400 MHz, DMSO-d₆) δ 9.53 (br, 1H) 7.35 (d, J=8.8 Hz, 2H), 6.84 (d, J=8.8 Hz, 2H), 4.40 (t, J=5.6 Hz, 1H), 4.17 (t, J=4.4 Hz, 2H), 3.91 (t, J =9.6 Hz, 2H), 3.24 (s, 3H), 3.23 (s, 6H), 1.71-1.63 (m, 4H).

Step F: Preparation of mPEG aldehyde B

mPEG aldehyde B was obtained as a white powder in 87% yield from mPEG Aldehyde B dimethyl acetal using the method described in Step F for preparing mPEG aldehyde A. ¹H NMR (400 MHz, DMSO-d₆) δ 9.71 (s, 1H), 9.54 (br, 1H), 7.34 (d, J=8.8 Hz, 2H), 6.83 (d, J=8.8 Hz, 2H), 4.17 (t, J=4.8 Hz, 2H), 3.91 (t, J=6.0 Hz, 2H), 3.24 (s, 3H), 2.60-2.56 (m, 2H), 1.97-1.93 (m, 2H).

Preparation of mPEG Aldehyde C:

Step A: Preparation of 3-(3-nitrophenoxy)propan-1-ol

3-(3-Nitrophenoxy)propan-1-ol was obtained as a pale yellow liquid in 93% yield from 3-nitrophenol and 3-chloropropan-1-ol using the method described in Step A for preparing mPEG aldehyde A. ¹H NMR (400 MHz, CDCl₃) δ 7.85 (d, J=8.0 Hz, 1H), 7.78 (s, 1H), 7.46 (t, J=8.0 Hz, 1H), 7.26 (d, J=8.0 Hz, 1H), 4.23 (t, J=6.0 Hz, 1H), 3.92 (t, J=6.0 Hz, 2H), 2.16-2.09 (m, 2H); ¹³C NMR (100 MHz, CDCl₃) δ 159.3, 149.1, 129.9, 121.5, 115.7, 108.7, 65.7, 59.6, 31.7.

Step B: Preparation of 3-(3-nitrophenoxy)propanal

3-(3-Nitrophenoxy)propanal was obtained as a pale yellow liquid in 78% yield from 3-(3-nitrophenoxy)propan-1-ol using the method described in Step B for preparing mPEG aldehyde A. ¹H NMR (400 MHz, CDCl₃) δ 9.90 (s, 1H), 7.85 (d, J=8.0 Hz, 1H), 7.75 (s, 1H), 7.45 (d, J=8.0 Hz, 1H), 7.26-7.22 (m, 1H), 4.40 (t, J=6.0 Hz, 2H), 2.99 (t, J=6.0 Hz, 2H); ¹³C NMR (100 MHz, CDCl₃) δ 199.1, 158.9, 149.1, 130.0, 121.5, 116.1, 108.7, 62.0, 42.8; GC-MS (m/z) calcd for C₉H₉NO₄: 195.2, found: 195, 167, 139, 93, 65.

Step C: Preparation of 3-(3-aminophenoxy)propanal dimethyl acetal

3-(3-Aminophenoxy)propanal dimethyl acetal was obtained as a deep purple liquid in 45% yield from 3-(3-nitrophenoxy)propanal using sequentially the method described in Step C for preparing mPEG aldehyde A and the method described in Step D for preparing mPEG aldehyde B. ¹H NMR (400 MHz, CDCl₃) δ 7.04 (t, J=8.0 Hz, 1H), 6.33-6.24 (m, 2H), 6.24 (s, 1H), 4.62 (t, J=5.6 Hz, 1H), 4.23 (t, J=4.4 Hz, 2H), 3.61 (br, 2H), 3.36 (s, 6H), 2.08-2.03 (m, 2H); ¹³C NMR (100 MHz, CDCl₃) δ 159.9, 147.6, 130.0, 107.9, 104.5, 102.1, 101.6, 63.6, 53.3, 32.8; GC-MS (m/z) calcd for C₁₁H₁₇NO₃: 211.2, found: 211, 196, 164, 148, 109, 75.

Step D: Preparation of mPEG aldehyde C dimethyl acetal

mPEG aldehyde C dimethyl acetal was obtained as a white powder in 95% yield from linear 20 kDa mPEG-OH and 3-(3-aminophenoxy)propanal dimethyl acetal using the method described in Step E for preparing mPEG aldehyde A. ¹H NMR (400 MHz, DMSO-d₆) δ 9.72 (br, 1H), 7.17-7.13 (m, 2H), 7.01 (d, J=8.0 Hz, 1H), 6.85 (d, J=8.0 Hz, 1H), 4.95 (t, J=5.6 Hz, 1H), 4.53 (t, J=4.8 Hz, 2H), 3.95 (t, J=9.6 Hz, 2H), 3.26 (s, 3H), 3.24 (s, 6H), 2.00-1.95 (m, 2H).

Step E: Preparation of mPEG aldehyde C

mPEG aldehyde C was obtained as a white powder in 95% yield from mPEG aldehyde C dimethyl acetal using the method described in Step F for preparing mPEG aldehyde A. ¹H NMR (400 MHz, DMSO-d₆) δ 9.72 (s, 1H), 9.69 (br, 1H), 7.20-7.13 (m, 2H), 7.01 (d, J =8.0 Hz, 1H), 6.55 (d, J=8.0 Hz, 1H), 4.24-4.07 (m, 4H), 3.24 (s, 3H), 2.87 (t, J=8.0 Hz, 2H).

Preparation of mPEG Aldehyde D:

Step A. Preparation of 4-(3-nitrophenoxy)butan-1-ol

4-(3-Nitrophenoxy)butan-1-ol was obtained in 81% yield from 3-nitrophenol and 2-[(4-chlorobutyl)oxy]tetrahydropyran using the method described in Step A for preparing mPEG aldehyde A, followed by reaction with concentrated sulfuric acid in ethanol at reflux for 0.5 hours. ¹H NMR (400 MHz, CDCl₃) δ 7.79 (d, J=8.0 Hz, 1H), 7.71 (s, 1H), 7.41 (t, J=8.0 Hz, 1H), 7.26-7.19 (m, 1H), 4.08 (t, J=6.0 Hz, 2H), 3.73 (t, J=6.4 Hz, 2H), 1.96-1.90 (m, 2H), 1.89-1.71 (m, 2H); GC-MS (m/z) calcd for C₁₀H₁₃NO₄: 211.2, found: 211, 139, 123, 109, 93, 73, 55.

Step B: Preparation of 4-(3-nitrophenoxy)butanal

4-(3-Nitrophenoxy)butanal was obtained in 78% yield from 4-(3-nitrophenoxy) butan-1-ol using the method described in Step B for preparing mPEG aldehyde A. ¹H NMR (400 MHz, CDCl₃) δ 9.86 (s, 1H), 7.82 (d, J=8.0 Hz, 1H), 7.71 (s, 1H), 7.42 (t, J=8.0 Hz, 1H), 7.22-7.19 (m, 1H), 4.09 (t, J=6.0 Hz, 2H), 2.70 (t, J=7.0 Hz, 2H), 2.20-2.14 (m, 2H).

Step C: Preparation of 4-(3-aminophenoxy)butanal dimethyl acetal

4-(3-Aminophenoxy)butanal dimethyl acetal was obtained in 52% yield from 4-(3-nitrophenoxy)butanal using sequentially the method described in Step C for preparing mPEG aldehyde A and the method described in Step D for preparing mPEG aldehyde B. ¹H NMR (400 MHz, CDCl₃) δ 7.10-7.04 (m, 1H), 6.94-6.33 (m, 3H), 4.43 (t, J=5.6 Hz, 1H), 3.92 (t, J=6.4 Hz, 2H), 3.34 (s, 6H), 1.82-1.78 (m, 4H); ¹³C NMR (100 MHz, CDCl₃) δ 160.1, 164.5, 130.1, 108.3, 105.3, 104.3, 102.1, 67.3, 52.8, 29.1, 24.5; GC-MS (m/z) calcd for C₁₂H₁₉NO₃: 225.3, found: 225, 194, 164, 109, 85.

Step D: Preparation of mPEG aldehyde D dimethyl acetal

mPEG aldehyde D dimethyl acetal was obtained as a white powder in 90% yield from linear 20 kDa mPEG-OH and 4-(3-aminophenoxy)butanal dimethyl acetal using the method described in Step E for preparing mPEG aldehyde A. ¹H NMR (400 MHz, DMSO-d₆) δ 9.71 (br, 1H), 7.16-7.12 (m, 2H), 7.01 (d, J=8.8 Hz, 1H), 6.54 (d, J=8.8 Hz, 1H), 4.95 (t, J=5.6 Hz, 1H), 4.20 (t, J=4.8 Hz, 2H), 3.92 (t, J=6.0 Hz, 2H), 3.25 (s, 6H), 3.24 (s, 3H), 1.71-1.64 (m, 4H).

Step E. Preparation of mPEG aldehyde D

mPEG aldehyde D was obtained as a white powder in 95% yield from mPEG aldehyde D dimethyl acetal using the method described in Step F for preparing mPEG aldehyde A. ¹H NMR (400 MHz, DMSO-d₆) δ 9.72 (s, 1H), 9.70 (br, 1H), 7.16-7.13 (m, 2H), 7.01 (d, J=8.8 Hz, 1H), 6.53 (d, J=8.8 Hz, 1H), 4.20 (t, J=4.4 Hz, 2H), 3.92 (t, J=6.0 Hz, 2H), 3.24 (s, 3H), 2.74-2.61 (m, 2H), 1.98-1.91 (m, 2H).

### EXAMPLE 2

### Preparation of Ser-Gly-IFN

A modified recombinant human interferon-α_{2b}, i.e., Ser-Gly-IFN, was cloned by a PCR method using human genomic DNA as a template. The oligonucleotides were synthesized based on the flanking sequences of human interferon-α_{2b} (GenBank Accession # NM_{—}000605). The derived PCR products were subcloned into pGEM-T vector (Promega). The IFN variant was PCR amplified again through the pGEM-T clones and subsequently subcloned into protein expression vector pET-24a (Novagen), a T7 RNA polymerase promoter driven vector, using NdeI/BamHI as the cloning sites. Vector pET-24a was then transformed into E. coli BL21-CodonPlus (DE 3)-RIL (Stratagene) strain. The high-expression clones were selected by maintaining the transformed E. coli BL21-CodonPlus (DE 3)-RIL at the presence of karamycin (50 μg/mL) and chloramphenical (50 μg/mL).

Terrific broth medium (BD, 200 mL) was employed for the propagation of BL21-CodonPlus (DE 3)-RIL with Ser-Gly-IFN gene in a 1,000 mL flask. The flask was shaken at 37° C. at 230 rpm for 16 hours. Batch and fed-batch fermentations were performed in a 5-liter jar fermentor (Bioflo 3000; New Brunswick Scientific Co., Edison, N.J.). The batch fermentation used 150 mL of an overnight preculture inoculum and 3 L of the Terrific broth medium with karamycin (50 ug/mL), chloramphenical (50 ug/mL), 0.4% glycerol, and 0.5% (v/v) trace elements (10 g/L of FeSO₄.7H₂O, 2.25 g/L of ZnSO₄.7H₂O, 1 g/L of CuSO₄.5H₂O, 0.5 g/L of MnSO₄.H₂O, 0.3 g/L of H₃BO₃, 2 g/L of CaCl₂.2H₂O, 0.1 g/L of (NH₄)₆Mo₇O₂₄, 0.84 g/L EDTA, 50 ml/L HCl). The dissolved oxygen concentration was controlled at 35% and the pH was kept at 7.2 by adding a 5 N NaOH aqueous solution. A feeding solution containing 600 g/L of glucose and 20 g/L of MgSO₄.7H₂O was prepared. When the pH rose to a value greater than the set point, an appropriate volume of the feeding solution was added to increase the glucose concentration in the culture broth. Expression of the Ser-Gly-IFN gene was induced by adding IPTG to a final concentration of 1 mM and the culture broth was harvested after incubating for 3 hours.

The collected cell pellet was resuspended with TEN buffer (50 mM Tris-HCl (pH 8.0), 1 mM EDTA, 100 mM NaCl) in an approximate ratio of 1:10 (wet weight g/mL) and disrupted by a microfluidizer, and then centrifuged at 10,000 rpm for 20 minutes. The pellet containing inclusion body (IB) was washed twice with TEN buffer and centrifuged as described above. The pellet containing IB was then suspended in 150 mL of a 4 M guanidium HCl (GuHCl) aqueous solution and centrifuged at 20,000 rpm for 15 minutes. The IB was then solubilized in 50 mL of 6 M GuHCl solution. The GuHCl solubilized material was centrifuged at 20,000 rpm for 20 minutes. Refolding was initiated by dilution of denatured IB in 1.5 L of a freshly prepared refolding buffer (100 mM Tris-HCl (pH 8.0), 0.5 M L-Arginine, 2 mM EDTA) that was stirred only during the addition. The refolding reaction mixture was allowed to incubate for 48 hours without stirring. The refolded recombinant human interferon-α_{2b} (i.e., Ser-Gly-IFN) was dialyzed against 20 mM Tris buffer (with 2 mM EDTA and 0.1M urea, pH 7.0) for further purification by Q-Sepharose column chromatography.

The refolded recombinant human protein Ser-Gly-IFN was loaded onto a Q-Sepharose column (GE Amersham Pharmacia, Pittsburgh, Pa.). The column was pre-equilibrated and washed with a 20 mM Tris-HCl buffer (pH 7.0). The product was eluted with a mixture of 20 mM Tris-HCl buffer (pH 7.0) and 200 mM NaCl. Fractions containing Ser-Gly-IFN was collected based on its absorbance at 280 nm. The concentration of Ser-Gly-IFN was determined by a protein assay kit using the Bradford method (Pierce, Rockford, Ill.).

### EXAMPLE 3

### Conjugation of mPEG Aldehyde A and Ser-Gly-IFN

A representative polypeptide-polymer conjugate involving mPEG Aldehyde A and Ser-Gly-IFN was prepared as follows:

The Q-Sepharose purified Ser-Gly-IFN (1 mg) prepared in Example 2 above was treated with mPEG aldehyde A. The final reaction mixture contained 50 mM sodium phosphate (pH 6.0), 5 mM sodium cyanoborohydride (Aldrich, Milwaukee, Wis.), and 10 mg of mPEG aldehyde A. The mixture was then incubated at room temperature for 20 hours to form as a major product the mono-PEGylated Ser-Gly-IFN, which was then purified by SP XL Sepharose chromatography (GE Amersham Pharmacia, Pittsburgh, Pa.). Specfically, the SP column was pre-equilibrated and washed with a solution of 20 mM sodium acetate (pH 5.4). Mono-PEGylated Ser-Gly-IFN was then eluted with a buffer containing 20 mM sodium acetate (pH 5.4) and 60 mM NaCl. The unreacted IFN, i.e., Ser-Gly-IFN, was eluted by a buffer containing 20 mM sodium acetate (pH 5.4) and 200 mM NaCl. The eluted fractions were analyzed by gel electrophoresis with a 12% sodium dodecyl sulfate-polyacrylamide gel and the signals were detected by staining with Coomassie brilliant blue R-250 and silver stain. Fractions containing mono-PEGylated Ser-Gly-IFN were collected based on their retention time and absorbance at 280 nm. The concentration of mono-PEGylated Ser-Gly-IFN was determined by a protein assay kit using the Bradford method (Pierce, Rockford, Ill.). The isolated yield of mono-PEGylated Ser-Gly-IFN was 30%-40%.

### EXAMPLE 4

### Physical and Biological Properties of mono-PEGylated Ser--Gly-IFN

The specificity of the pegylation reaction above was determined by tryptic peptide mapping of both Ser-Gly-IFN and mono-PEGylated Ser-Gly-IFN. A 100 μg sample of each compound was vacuum dried and reconstituted in 60 μL of a 8 M urea/0.4 M NH₄HCO₃ solution. After treated with reducing agents and iodoacetic acid, the solutions were digested with trypsin from Promega (sequencing grade). Aliquots were taken and injected into a C18 HPLC column. The resulting tryptic peptides were separated using a 75-min gradient eluant containing from 0 to 70% acetonitrile in 0. 1% TFA-H₂O. The peptide fragments from both the Ser-Gly-IFN and mono-PEGylated Ser-Gly-IFN samples were monitored by their absorbance at 214 nm and were manually collected, dried by a Speed-Vac system, and subjected to MALDI-TOF analysis. Comparison of the data from both samples indicated that the major site of the pegylation reaction occurred at the N-terminus of Ser-Gly-IFN.

The antiviral activities of mono-PEGylated Ser-Gly-IFN and the mono-PEGylated products of other modified human IFN-α_{2b} variants (i.e., mono-PEGylated -Gly-Ser-IFN, -Met-Met-IFN, -Met-His-IFN, -Pro-IFN, and -Gly-Met-IFN) were tested on Bovine kidney epithelium cells (MDBK) challenged by vesicular stomatitis virus (VSV). The cytopathic effect (CPE) of the infected cells was determined by the formation of formazan from the viable cellular enzymes after the addition of tetrazolium salt WST-1 into the assay. This CPE bioassay was performed using triplicate data points for each concentration. The specific antiviral activities of all these mono-PEGylated modified human IFN-α_{2b} compounds were calculated based on the concentration that provides 50% of cellular protection (EC₅₀, i.e., 50% of cytopathic effects). The results of CPE antiviral bioassay were reported in units of IU/mg using Roferon® as a reference standard. The results show that the CPE bioactivity of mono-PEGylated Ser-Gly-IFN were 2.0×10⁸ and the CPE bioactivity of other mono-PEGylated human IFN-α_{2b} variants range from 8.3×10⁶ to 2.9×10⁷ IU/mg.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A polypeptide-polymer conjugate comprising:
a polypeptide moiety;
a polyalkylene oxide moiety;
a linker connecting the polypeptide moiety with the polyalkylene oxide moiety;
a first linkage between the polypeptide moiety and the linker; and
a second linkage between the polyalkylene oxide moiety and the linker; wherein the polypeptide moiety contains a human interferon-α moiety and 1-6 additional amino acid residues at the N-terminus of the human interferon-α moiety; the polyalkylene oxide moiety contains 1-20,000 C₁-C₈ alkylene oxide repeating units; the linker is C₁-C₈ alkylene, C₁-C₈ heteroalkylene, C₃-C₈ cycloalkylene, C₃-C₈ heterocycloalkylene, arylene, heteroarylene, aralkylene, or —Ar—X—(CH₂)ₙ—, in which Ar is arylene or heteroarylene, X is O, S, or N(R), R being H or C₁-C₁₀ alkyl, and n is 1-10; and each of the first and second linkages, independently, is a carboxylic ester, carbonyl, carbonate, amide, carbamate, urea, ether, thio, sulfonyl, sulfinyl, amino, imino, hydroxyamino, phosphonate, or phosphate group.

2. The conjugate of claim 1, wherein the human interferon-α moiety is a human interferon-α_{2b} moiety.

3. The conjugate of claim 2, wherein the polypeptide moiety is -Ser-Gly-IFN, in which IFN is the human interferon-α_{2b} moiety.

4. The conjugate of claim 3, wherein the polyalkylene oxide moiety is a polyethylene oxide moiety containing 5-10,000 repeating units.

5. The conjugate of claim 4, wherein the polyethylene oxide moiety has a number average molecular weight of 20,000 Daltons.

6. The conjugate of claim 5, wherein the linker is —Ar—X—(CH₂)ₙ—.

7. The conjugate of claim 6, wherein Ar is phenylene.

8. The conjugate of claim 7, wherein X is O.

9. The conjugate of claim 8, wherein n is 3.

10. The conjugate of claim 9, wherein the first linkage is an amino group and the second linkage is a carbamate group.

11. The conjugate of claim 10, wherein the conjugate is 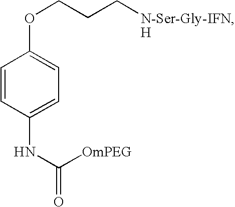
in which mPEG is a methoxy-capped polyethylene oxide moiety.

12. The conjugate of claim 1, wherein the polyalkylene oxide moiety is a polyethylene oxide moiety containing 5-10,000 repeating units.

13. The conjugate of claim 12, wherein the polyethylene oxide moiety has a number average molecular weight of 20,000 Daltons.

14. The conjugate of claim 1, wherein the linker is —Ar—X—(CH₂)ₙ—.

15. The conjugate of claim 14, wherein Ar is phenylene.

16. The conjugate of claim 15, wherein X is O.

17. The conjugate of claim 16, wherein n is 3.

18. The conjugate of claim 1, wherein the first linkage is an amino group and the second linkage is a carbamate group.

19. The conjugate of claim 1, wherein the human interferon-α moiety has a cysteine residue at the N-terminus.

20. A polypeptide-polymer conjugate comprising:
a polypeptide moiety;
a polyalkylene oxide moiety;
a linker connecting the polypeptide moiety with the polyalkylene oxide moiety;
a first linkage between the polypeptide moiety and the linker; and
a second linkage between the polyalkylene oxide moiety and the linker; wherein the polyalkylene oxide moiety contains 1-20,000 C₁-C₈ alkylene oxide repeating units; the linker is —Ar—X—(CH₂)ₙ—, in which Ar is arylene or heteroarylene, X is O, S, or N(R), R being H or C₁-C₁₀ alkyl, and n is 1-10; and each of the first and second linkages, independently, is a carboxylic ester, carbonyl, carbonate, amide, carbamate, urea, ether, thio, sulfonyl, sulfinyl, amino, imino, hydroxyamino, phosphonate, or phosphate group.

21. The conjugate of claim 20, wherein Ar is phenylene.

22. The conjugate of claim 21, wherein X is O.

23. The conjugate of claim 22, wherein n is 3.

24. The conjugate of claim 20, wherein the polypeptide moiety contains an interferon-α moiety and 1-6 additional amino acid residues at the N-terminus of the interferon-α moiety.

25. The conjugate of claim 20, wherein the polypeptide moiety contains an interferon-β moiety or a granulocyte colony-stimulating factor.

26. A compound of formula (I): 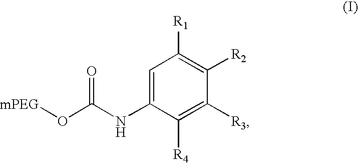
wherein
mPEG is a methoxy-capped polyethylene oxide moiety;
one of R₁, R₂, R₃, and R4 is C₁-C₁₀ alkyl substituted with CHO; and
each of the other R₁, R₂, R₃, and R₄, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl.

27. The compound of claim 26, wherein the mPEG contains 5-10,000 repeating units.

28. The compound of claim 27, wherein the mPEG has a number average molecular weight of 20,000 Daltons.

29. The compound of claim 26, wherein R₂ is propyl substituted with CHO or butyl substituted with CHO.

30. The compound of claim 26, wherein R₃ is propyl substituted with CHO or butyl substituted with CHO.

31. A polypeptide comprising an interferon-α moiety and 1-6 additional amino acid residues at the N-terminus of the interferon-α moiety.

32. The polypeptide of claim 31, wherein the interferon-α moiety is a human interferon-α moiety.

33. The polypeptide of claim 32, wherein the human interferon-α moiety is a human interferon-α_{2b} moiety.

34. The polypeptide of claim 33, wherein the human interferon-α moiety has a cysteine residue at the N-terminus.

35. The polypeptide of claim 34, wherein the human interferon-α moiety is a wild type interferon-α moiety.

36. The polypeptide of claim 31, wherein the polypeptide is Ser-Gly-IFN, Gly-Ser-IFN, Met-Met-IFN, Met-His-IFN, Pro-IFN, or Gly-Met-IFN, in which IFN is a human interferon-α_{2b} moiety.

37. The polypeptide of claim 31, wherein the interferon-α moiety is a wild type interferon-α moiety.
